(19) United States
(12) Patent Application Publication
(10) Pub. No.: US 2026/0308689 A1
(43) Pub. Date: Oct. 8, 2026

(54) DETECTION OF TUMOR-DERIVED NUCLEIC ACIDS USING RED BLOOD CELLS

(71) Applicant: THE TRUSTEES OF THE UNIVERSITY OF PENNSYLVANIA, Philadelphia, PA (US)

(72) Inventors: Nilam Mangalmurti, Bryn Mawr, PA (US); Jeffrey Thompson, Merion Station, PA (US)

(21) Appl. No.: 19/481,409

(22) PCT Filed: May 6, 2024
(86) PCT No.: PCT/US2024/028028

§ 371 Date: Nov. 4, 2025

Related U.S. Application Data
(60) Provisional application No. 63/500,136, filed on May 4, 2023.
(60) Provisional application No. 63/636,309, filed on Apr. 19, 2024.

(51) Int. Cl. C12Q 1/6886; C12Q 1/48; C12Q 1/6827; C12Q 1/6851; C12Q 1/686

(52) U.S. Cl. CPC C12Q 1/6886; C12Q 1/48; C12Q 1/6827; C12Q 1/6851; C12Q 1/686; C12Y 207/07; C12Q 2600/156; C12Q 2600/16; G01N 2800/52; G01N 2800/7028

(57) ABSTRACT

Described herein are methods of diagnosing a cancer in a subject. The method includes contacting a red blood cell-containing sample from the subject with a reagent capable of detecting a tumor-associated molecule in the sample; and diagnosing the subject with a tumor when the tumor-associated molecule is detected in the sample. In some embodiments, suitable samples are less than 10 μL.

### CROSS-REFERENCE TO RELATED APPLICATIONS

This application claims priority of U.S. Provisional Application No. 63/500,136 filed May 4, 2023, and U.S. Provisional Application No. 63/636,309, filed Apr. 19, 2024, the entire contents of each being incorporated herein by reference as though set forth in full.

### INCORPORATION-BY-REFERENCE OF MATERIAL SUBMITTED IN ELECTRONIC FORM

The content of the electronic sequence listing (UPN-22-10081-PCT.xml; Size: 45,266 bytes; and Date of Creation: May 6, 2024) is herein incorporated by reference in its entirety.

### BACKGROUND OF THE INVENTION

Lung cancer is the leading cause of cancer-related death worldwide, with a 5-year survival of only 23%. The profiling of circulating tumor cell-free DNA (ctDNA) has emerged as a promising tool for biopsy-free tumor genotyping, monitoring disease burden, and early cancer detection. ctDNA consists of short DNA fragments shed into the bloodstream by tumor cells. It can be analyzed using highly sensitive sequencing assays to enable the detection of tumor-specific mutations, structural variants, and copy number alterations in patients with lung cancer. Analysis of ctDNA is being increasingly utilized for non-invasive mutation detection to guide targeted therapy selection in patients with advanced disease. However, ctDNA has a short half-life of only 1-2 hours in circulation and generally represents a very small fraction of the total circulating cell-free DNA, thus substantially limiting the sensitivity and clinical utility of these assays.

A need in the art exists for improved methods for detecting tumor DNA in patient samples.

### SUMMARY OF THE INVENTION

Provided herein, in a first aspect, is a method of diagnosing cancer in a subject. The method includes contacting a red blood cell-containing sample from the subject with a reagent capable of detecting a tumor-associated molecule in the sample and diagnosing the subject with a cancer when the tumor-associated molecule is detected in the sample. In certain embodiments, the reagent is specific to a tumor-associated genetic mutation. In certain embodiments, the reagent comprises multiple reagents, each capable of detecting a different specific tumor-associated molecule. In certain embodiments, the reagent comprises primers capable of binding tumor-associated DNA or RNA. In certain embodiments, the tumor-associated genetic mutation is found in the EGFR, ALK, KRAS, ROS1, BRAF, NTRK, MET, RET, ERBB2, PIK3CA, BRCA1, BRCA2, FGFR2, FGFR3, EZH2, or PDGFRA gene.

In other aspects, compositions and kits for performing the methods described herein are provided.

### BRIEF DESCRIPTION OF THE DRAWINGS

FIG. 1A-1H show RBCs acquire tumor DNA from lung cancer cells. Lung cancer cells (A549 cells bearing the G12S mutation) were seeded in 6 well plates at 1×10⁶ cells/well density. The cells were grown to confluence and then incubated for 24 or 72 hours with highly purified RBCs at the specified dose. The cell culture supernatant was then aspirated, and RBCs were isolated by centrifugation. The RBCs were subsequently washed 2 times before cryopreservation at −80° C. DNA was extracted from the frozen RBCs as we have previously described by our group and qPCR was performed using primers specific for G12 S (A and D), commercially available mutant multiplex primers (B&E) or Universal KRAS primers. G. Summation of the 24-hour and 72-hour co-culture studies. H. DNA gel demonstrating the G12S amplicon present in the A549 cells or RBCs treated with A549 cells, the expected amplicon size is 98 bp. As seen in the figure above, RBCs do not contain KRAS DNA under basal conditions but acquire the DNA from tumor cells. Data in FIG. 1A-1G, is presented as Cycle threshold (Ct) with the lower Ct indicating more abundant DNA. Technical replicates are presented in FIG. 1.

FIG. 2A-2C show allele specific primers are specific and do not amplify in control cell lines. Lung cancer cells (A549 cells bearing the G12S mutation, H358 cells bearing the G12C mutation or H1299 cells) were seeded in 6 well plates. The following day the cells were treated with varying doses of purified RBCs. The cell culture supernatant was then aspirated, and RBCs were isolated by centrifugation. The RBCs were subsequently washed 2 times before cryopreservation at −80° C. DNA was extracted from the frozen RBCs as we have previously described by our group and qPCR was performed using allele specific primers (G12S or G12C), commercially available mutant multiplex primers or Universal KRAS primers. A summary heatmap of all the experimental conditions is provided below the bar graph. As seen in the figure above, RBCs do not contain KRAS DNA under basal conditions but acquire the DNA from tumor cells. Data is presented as Cycle threshold (Ct) with the lower Ct indicating more abundant DNA.

FIG. 3A-3C show RBCs sequester tumor cell DNA. Differing volumes of lung cancer cell lysate (A549, H358, and H1299) was incubated with varying amounts of RBCs for 2 hours. The RBC pellet was subsequently isolated from the supernatant (SN) via sucrose density centrifugation. DNA was extracted from the supernatant (200 uL) or the RBC pellet (10⁷) and qPCR was run using allele specific primers or Universal KRAS primers. As seen in the figure the RBCs sequester tumor DNA. The data is presented as Ct v volume of tumor lysate, corresponding heatmaps for each experiment are also shown.

FIGS. 4A and 4B shows RNA content is different between healthy and patient RBCs. RBCs were magnetically purified using glycophorin A-conjugated beads. RNA extracted from 107 RBC using Zymo Quick DNA/RNA Microprep kit were analyzed by TapeStation. (FIG. 4A) Electrophoresis of RNA from RBCs and (FIG. 4B) corresponding signal intensity profiles in two of the samples. EL1(L): electronic ladder, nt: nucleotide(s).

FIG. 5A-5D demonstrates that RBCs from patients with early stage lung cancer are positive for tumor DNA. FIG. 5A and FIG. 5B. qPCR for the mutant KRAS G12C using mutant specific primers was performed on RBC samples from patients with lung cancer (patient 1—Stage 1A, patient 2—Stage IIB). The amplification curves are shown in FIG. 5A and FIG. 5B. Both patient samples demonstrate the presence of the KRAS G12C tumor DNA. FIG. 5C. the cycle threshold for the RBC samples from patients, positive control (tumor cell lysate from the H358 line that bears the G12 C mutation) and negative control are shown. The lower the Ct, the more DNA is present in the sample. FIG. 5D. a gel of the PCR products was run. The lanes are as follows, blank—marker, 1—RBCs from patient 1, 2—RBC sample from a patient with stage IIIA lung cancer, 3—RBC sample from another patient with stage IIIA lung cancer 4—RBCs from patient 2, 5—RBCs from a patient with stage 1A (not known to be positive for G12C), 6—RBCs from a patient with stage 1A. 7—buffer. 8—positive control, H358 cell lysate.

FIG. 6 shows comparison of wild type KRAS amplification on plasma and RBCs. DNA was extracted from 5 uL of RBCs or plasma and qPCR was run for KRAS (universal primers that do not detect mutant KRAS were used). As seen in the figure, the RBCs amplify earlier than plasma, suggesting that the DNA is sequestered by the RBCs.

FIG. 7 shows a diagram representing experiment performed in Example 6.

FIGS. 8A-8B show the amount of Human Papillomavirus (HPV) DNA that binds to RBCs when incubated with various amounts of HPV CpG.

FIG. 9 shows a diagram representing experiment performed in Example 7.

FIG. 10 shows an HPV16 qPCR amplification curve.

FIG. 11 shows a graphical representation and a HPV16qPCR gel indicating that RBCs acquire HPV16 DNA from cervical cancer cell lines.

FIG. 12 shows a copy number analysis of cervical cancer samples.

FIG. 13A-13C show healthy donor RBCs acquire HPV DNA and HPV DNA is detectable on RBCs from patients with locally advanced HPV 16+ cervical cancer. RBCs from 4 distinct healthy donors were incubated with the cancer cell line (CaSki cells, an HPV 16 positive cell line) for 24 hours. Following incubation the DNA was extracted from CaSki cell supernatant, RBCs incubated with Caski cells (1e7+ CaSki) or RBCs alone (1e7 alone). Filtered cell free culture media and water served as positive and negative controls. DNA was also extracted from differing amounts of CaSki cells. As seen in FIG. 13A, RBCs alone do not express HPV but HPV is detectable on RBCs cultured with CaSki cells. 5 independent experiments across 4 healthy donors. Points on the graph represent mean Ct from individual experiments. **** p<0.0001, one way ANOVA with Sidak's multiple comparisons test. FIG. 13B. RBCs from two patients with locally advanced cervical cancer were tested for the presence of HPV DNA (Gyn 10 and Gyn 11). As seen on the graph, both patients RBCs demonstrate detectable HPV DNA (2e7 and 10 uL volumes). FIG. 13C. Corresponding DNA gel demonstrating the presence of HPV DNA on the RBCs of patients but not in the plasma.

FIG. 14 analysis of RBV binding and sequestering of CpG via TLR9.

### DETAILED DESCRIPTION OF THE INVENTION

The ability to employ red blood cells (RBCs) to optimize the detection of rare ctDNA fragments is paradigm shifting and impacts early cancer detection, enabling more personalized therapeutic strategies. RBCs comprise the majority of circulating cells in mammals and are essential for respiration. Although non-gas exchanging functions of the red cell such as chemokine regulation, complement binding and pathogen immobilization have been described, the immune function of RBCs is not fully understood. RBCs transit through all tissues and are in contact with pathogen and self-derived inflammatory mediators in the circulation, positioning them as ideal messengers between remote organs. Recently, an immune role of RBCs as nucleic acid sensors has been uncovered and we have found that human RBCs can sequester DNA and RNA through the expression of toll-like receptors (TLRs).

The discovery of RBCs as a nucleic acid reservoir is paradigm-shifting. Improved ctDNA detection could revolutionize the diagnostic approach to ctDNA analysis. In addition, this technology could have broad implications for managing patients with lung cancer, including facilitation of early detection of disease, detection of minimal residual disease, assessing prognosis, and development of personalized therapeutic strategies.

Described herein are methods of detecting cancer. Our data demonstrate that RBCs are a rich source of tumor-derived nucleic acids. Furthermore, using RBC-based nucleic acid amplification, we can detect tumors using less than a drop of blood. We demonstrate that RBCs can be used to detect lung tumors.

By the terms “patient” or “subject” as used herein is meant a mammalian animal, including a human, a veterinary or farm animal, a domestic animal or pet, and animals normally used for clinical research, including non-human primates, dogs and mice. More specifically, the subject of these methods is a human. In one embodiment, the subject is suspected of having a tumor, or a complication therefrom.

### Sample

All of the compositions, kits and methods described herein rely on the observation that RBC-containing samples can be used to detect, diagnose, treat and help predict outcome of various tumors.

As used herein, the term “sample” or “patient sample” refers to a biological sample derived from a subject which contains red blood cells (RBCs). Also known as erythrocytes, RBCs are the most common type of cell found in the blood, with each cubic millimeter of blood containing 4-6 million cells.

In certain embodiments, the sample is substantially free from all other blood components other than RBCs. Thus, in one embodiment, a sample of whole blood is obtained from a subject, and RBCs are isolated, concentrated or purified. In one embodiment, the sample is filtered to remove non-RBCs, and RBCs are isolated from the sample based on size. RBCs have a diameter of about 6-8 μM.

Another advantage of the compositions, methods, and kits described herein, is the small volume of sample required. In some embodiments, the sample is a drop of blood, which can be obtained from a finger stick at the point of care. Prior art methodologies require a venous blood draw, which is more invasive, more expensive, and requiring of specialized equipment and training. In contrast, in some embodiments, the compositions, methods and kits described herein require only about 1 μL to about 10 μL of blood, or a drop of blood or less. In one embodiment, the sample is about 1 μL, 2 μL, 3 μL, 4 μL, 5 μL, 6 μL, 7 μL, 8 μL, 9 μL, or 10 μL. In another embodiment, the sample is about 10 μL, 11 μL, 12 μL, 13 μL, 14 μL, 15 μL, 16 μL, 17 μL, 18 μL, 19 μL, or 20 μL. In yet another embodiment, the sample is about 10 μL or less. In another embodiment, the sample volume is about 1 μL to about 20 μL. In another embodiment, the sample volume is about 1 μL to about 10 μL. In another embodiment, the sample volume is about 2 μL to about 5 μL. Each of these ranges includes endpoints and all integers therebetween.

In another embodiment, the sample is less than about 1 mL. In another embodiment, the sample is about 100 μL, 200 μL, 300 μL, 400 μL, 500 μL, 600 μL, 700 μL, 800 μL, 900 μL, 1 mL, including all integers therebetween.

The sample must contain a sufficient number of red blood cells. In one embodiment, the sample contains at least 1 million RBCs. In another embodiment, the sample contains at least 1.5 million, 2 million, 2.5 million, 3 million, 3.5 million, 4 million, 4.5 million, 5 million, 5.5 million, 6 million, 6.5 million, 7 million, 7.5 million, 8 million, 8.5 million, 9 million, 9.5 million, or 10 million RBCs. In yet another embodiment, the sample contains at least 20 million, 30 million, 40 million, 50 million, 60 million, 70 million, 80 million, 90 million, or 100 million RBCs. In another embodiment, the sample contains at most 1.5 million, 2 million, 2.5 million, 3 million, 3.5 million, 4 million, 4.5 million, 5 million, 5.5 million, 6 million, 6.5 million, 7 million, 7.5 million, 8 million, 8.5 million, 9 million, 9.5 million, or 10 million RBCs. In yet another embodiment, the sample contains at most 20 million, 30 million, 40 million, 50 million, 60 million, 70 million, 80 million, 90 million, or 100 million RBCs. In another embodiment, the sample contains about 1.5 million, 2 million, 2.5 million, 3 million, 3.5 million, 4 million, 4.5 million, 5 million, 5.5 million, 6 million, 6.5 million, 7 million, 7.5 million, 8 million, 8.5 million, 9 million, 9.5 million, or 10 million RBCs. In yet another embodiment, the sample contains about 20 million, 30 million, 40 million, 50 million, 60 million, 70 million, 80 million, 90 million, or 100 million RBCs.

In some embodiments, a sample is obtained from a subject and treated to purify or enrich the sample for RBC. For example, the sample may be filtered to remove components smaller than and/or larger than an RBC, which is about 6-8 μM in diameter. The sample may also be sorted by density of the blood components, with the RBC component being isolated for use as described herein.

### Tumors

The compositions, methods and kits described herein are, in some embodiments, used to detect and identify cancerous tumors. As used herein the term “cancer” refers to or describes the physiological condition in mammals that is typically characterized by unregulated cell growth. In one embodiment, the term “cancer” means any cancer characterized by the presence of a solid tumor. In another embodiment, a cancer is a hematological cancer. When referred to herein, a cancer includes, without limitation, melanoma, cervical cancer, breast cancer, brain cancer, colon/rectal cancer, ovarian cancer, adrenal cancer, anal cancer, bile duct cancer, bladder cancer, bone cancer, endometrial cancer, esophagus cancer, eye cancer, kidney cancer, laryngeal cancer, liver cancer, head and neck cancer, nasopharyngeal cancer, osteosarcoma, oral cancer, ovarian cancer, pancreatic cancer, prostate cancer, rhabdomyosarcoma, salivary gland cancer, stomach cancer, testicular cancer, thyroid cancer, vaginal cancer, lung cancer, lymphoma, myeloma, and neuroendocrine cancer. In certain embodiments, the cancer is cervical cancer caused by human papillomavirus.

In certain embodiments, the compositions, methods, and kits include a reagent/reagents capable of detecting a tumor-associated molecule, e.g., tumor-associated genetic mutation. In certain embodiments, the genetic mutation is found in one of the genes listed in Table 1.

TABLE 1

| Gene | Genbank Ref Number |
|---|---|
| EGFR | NG_007726.3 |
| ALK | NP_004295 |
| KRAS | NP_203524 |
| ROS1 | NP_002935 |
| BRAF | NP_004324.2 |
| NTRK | NP_001012331 |
| MET | NP_001120972.1 |
| RET | NP_066124.1 |
| ERBB2 | NP_004439.2 |
| PIK3CA | NP_006209.2 |
| BRCA1 | AAC37594.1 |
| BRCA2 | AAB07223.1 |
| FGFR2 | CAA96492.1 |
| FGFR3 | CAA96492.1 |
| EZH2 | NP_004447.2 |
| PDGFRA | NP_006197.1 |

In certain embodiments, the tumor-associated molecule is derived from a tumor-associated genetic mutation in the gene EGFR. In another embodiment, the tumor-associated molecule is derived from a tumor-associated genetic mutation in the gene ALK. In certain embodiments, the tumor-associated molecule is derived from a tumor-associated genetic mutation in the gene KRAS. In certain embodiments, the tumor-associated molecule is derived from a tumor-associated genetic mutation in the gene ROS1. In certain embodiments, the tumor-associated molecule is derived from a tumor-associated genetic mutation in the gene BRAF. In certain embodiments, the tumor-associated molecule is derived from a tumor-associated genetic mutation in the gene NTRK. In certain embodiments, the tumor-associated molecule is derived from a tumor-associated genetic mutation in the gene MET. In certain embodiments, the tumor-associated molecule is derived from a tumor-associated genetic mutation in the gene RET. In certain embodiments, the tumor-associated molecule is derived from a tumor-associated genetic mutation in the gene ERBB2. In certain embodiments, the tumor-associated molecule is derived from a tumor-associated genetic mutation in the gene BRCA1. In certain embodiments, the tumor-associated molecule is derived from a tumor-associated genetic mutation in the gene BRCA2. In certain embodiments, the tumor-associated molecule is derived from a tumor-associated genetic mutation in the gene FGFR2. In certain embodiments, the tumor-associated molecule is derived from a tumor-associated genetic mutation in the gene FGFR3. In certain embodiments, the tumor-associated molecule is derived from a tumor-associated genetic mutation in the gene PDGFRA. The nucleic acid and amino acid sequences of the genes of Table 1 are known in the art, and are incorporated by reference herein.

For the identified genes, one or more mutations from the native coding and/or protein sequence are known to be associated with cancer. For example, KRAS mutations are present in approximately 25% of tumors, making them one of the most common gene mutations linked to cancer. Several KRAS mutations are known, including KRAS G12C, G12D, and G12R. Using the method described herein, it is possible to detect a specific KRAS mutation without the need for a tumor biopsy. Tables 2 and 3 provide known mutations for some of the tumor-associated genes described herein.

TABLE 2
KRAS mutations
Mutations in KRAS

10570G > T/G > C/G > A--> Gly12Cys/GLY12ARG/Gly12Ser
10571G > C/G > T/G > A--> Gly12Ala/GLY12Val/Gly12Asp
10573G > T/G > C/G > A --> Gly13Cys/Gly13Arg/Gly13Ser
10574G > A/G > C/G > T --> Gly13Ala/Gly13Asp/Gly13Val
28572G > A/Ala59Thr
28578C > A-->Gln61Lys
28579A > T/A > G-->Gln61Leu/Gln > Arg
28580A > C/A > T-->GIn61His
30208A > C/A > T-->Lys117Asp)
30293G > C/G > A-->Ala146Pro/Ala146Thr
30294C > T-->Ala146Val

TABLE 3
Mutations of tumor-associated genes

| Mutations | Mutations |
|---|---|
| EGFR_rf | KRAS c.35G > C p.Gly12Ala |
| EGFR c.2238_2252del15 | KRAS c.34G > C p.Gly12Arg |
| p.L747_T751delLREAT | |
| EGFR c.2573T > G p.Leu858Arg; EGFR | KRAS c.436G > A p.Ala146Thr |
| c.2572_2573CT > AG p.Leu858Arg | |
| EGFR c.2369C > T p.Thr790Met | KRAS c.37G > T p.Gly13Cys |
| EGFR c.2582T > A p.Leu861Gln | KRAS c.183A > C p.Gln61H |
| c.2303G > T p.Ser768Ile | KRAS c.182A > T p.Gln61Leu |
| EGFR c.2156G > C Gly719Ala | KRAS c.37G > C p.Gly13Arg |
| EGFR c.2155G > A p.Gly719Ser | NRAS_rf |
| EGFR c.2155G > T P.Gly719Cys | NRAS c.37G > C p.Gly13Arg |
| EGFR c.2125G > A p.Glu709Lys | NRAS c.34G > T p.Gly12Cys |
| EGFR c.2126A > C p.Glu709Ala | NRAS c.34G > A p.Gly12Ser |
| EGFR c.2311_2312insGCGTGGACA | NRAS c.38G > A p.Gly13Asp |
| EGFR c.2319_2320ins9 | NRAS c.182A > T p.Gln61Leu; NRAS |
| | c.181_182CA > TT p.Gln61Leu |
| BRAF_rf | NRAS c.35G > A p.Gly12Asp |
| BRAF c.1799T > A p.Val600Glu | NRAS c.181C > A p.Gln61Lys |
| BRAF c.1798_1799GT > AA p. Val600Glu | NRAS c.182A > G p.Gln61Arg; NRAS |
| | c.181_182CA > AG p.Gln61Arg |
| BRAF c.1798_1799GT > AG p.Val600Arg | NRAS c.38G > T p.Gly13Val |
| BRAF c.1799_1800TG > AT p.Val600Asp | NRAS c.35G > T p.Gly12Val |
| KRAS_rf | NRAS c.183A > T p.Gln61His |
| KRAS c.35G > A p.Gly12Asp | NRAS c.183A > C p.Gln61His |
| KRAS c.35G > T p.Gly12Val | NRAS c.35G > C p.Gly12Ala |
| KRAS c.38G > A p.Gly13Asp | NRAS c.37G > T p.Gly13Cys |
| KRAS c.34G > T p.Gly12Cys | NRAS c.37G > A p.Gly13Ser |
| KRAS c.34G > A p.Gly12Ser | NRAS c.38G > C p.Gly13Ala |

Methods of Detection and/or Diagnosis

Provided herein are methods of detecting tumors in a sample from a subject. The methods include contacting a red blood cell-containing sample from a subject with a reagent capable of detecting a tumor-associated molecule in the sample. In one embodiment, the subject is diagnosed with a tumor when the tumor-associated molecule is detected in the sample. In certain embodiment, the method further includes treating the subject when the tumor-associated molecule is detected. In certain embodiments, the tumor is from a cervical cancer. In certain embodiments, the tumor is from a cervical cancer and the tumor-associated molecule is a nucleotide from human papillomavirus (HPV).

In one embodiment, the reagent is able to detect the tumor by forming a complex with a tumor-associated molecule in the sample. In one embodiment, the subject is diagnosed with a tumor when the complex comprised of the tumor-associated molecule and reagent is detected in the sample. In yet another embodiment, the reagent is capable of amplifying the tumor-associated molecule, or product thereof. As used herein, the term “tumor-associated molecule” refers to any biological molecule that is derived from, or indicates the presence of, a tumor. Such molecules include nucleic acids (e.g., DNA, RNA, mRNA, etc.) and proteins found in or on the tumor, including surface proteins (e.g., glycoproteins, spike proteins, capsid proteins, F protein, G protein, etc.), antibodies elicited by the tumor, and mRNA transcripts. The presence of tumor-associated molecules indicates the presence of the tumor in the subject.

Various methods and techniques are known for detecting, amplifying or binding a tumor-associated molecule in a sample. Such methods include nucleic acid-based methods (e.g., PCR-based methods) and protein-based methods (e.g., ELISA or flow cytometery). The reagents described herein are specific to a tumor-associated genetic mutation. By specific to a tumor-associated genetic mutation, it is meant that the reagent binds, identifies or amplifies a particular nucleic acid containing the genetic mutation. For example, in one embodiment, for the KRAS G12C mutation, the reagents are capable of detecting the specific codon change that corresponds to the G12C mutation in DNA or mRNA transcripts found in the sample. Reagents such as these are known in the art, or can be designed by one skilled in the art, in view of the nucleic acid/amino acid sequence of the gene/protein. For example, see Guarnaccia M, Iemmolo R, San Biagio F, Alessi E, Cavallaro S. Genotyping of KRAS Mutational Status by the In-Check Lab-on-Chip Platform. Sensors (Basel). 2018 Jan. 5; 18(1):131, which describes KRAS mutation genotyping using PCR. This reference is incorporated herein by reference.

Alternatively, in another embodiment, the reagent is capable of detecting any mutation at a specific position or positions in a nucleic acid. These examples are not meant to limit the term “tumor-specific” reagent, which is meant to encompass any reagent which can discriminate between two or more tumors. In one embodiment, the reagent described herein comprises multiple reagents, where each reagent capable of detecting a different specific tumor. In one embodiment, 2, 3, 4, 5, 6, 7, 8, 9, 10, 11, 12, 13, 14, 15, 16, 17, 18, 19, 20, 21, 22, 23, 24, 25, 30, 25, 40, 45, 50, 55, 60, 65, 70, 75, 80, 85, 90, 95, 100 or more different reagents are contained in the composition or utilized in the methods.

In one embodiment, the reagent is capable of detecting, binding, specifically complexing with, or measuring the level of a tumor-associated molecule when present in the sample. In one embodiment, the reagents are those which are capable of detecting or measuring the amount or level of a tumor using nucleic acids, e.g., DNA or RNA. Kits for performing the same are available commercially, including, without limitation, Competitive Allele-Specific TaqMan® polymerase chain reaction (CastPCR) (Applied Biosystems). CastPCR has a high sensitivity and specificity; its allele-specific primer and locus-specific primer guarantee the amplification of the mutant allele, while an oligonucleotide blocker suppresses amplification of the wild type allele. Exemplary mutations that can be detected include those in Tables 2 and 3.

The diagnostic reagent may be a polynucleotide or genomic probe that hybridizes to the tumor DNA or RNA. Such polynucleotides may be about 20, about 22, about 25 or more nucleotides in length. In another embodiment, the diagnostic reagent is a PCR primer-probe set that amplifies and detects a polynucleotide sequence of the suspected tumor. In one embodiment, the reagent is immobilized on a substrate. In another embodiment, the diagnostic reagent comprises a microarray, a microfluidics card, a computer-readable chip or chamber. Suitable assays utilizing the described polynucleotide, genomic probe, or a pair of PCR primers may include but are not limited to PCR, reverse-transcriptase PCR, quantitative PCR, southern blot analysis, dot-blot hybridization and fluorescence in situ hybridization (FISH). Conventional methods or tools can be utilized by one of skill in the art in designing suitable polynucleotide, genomic probe, or a pair of PCR primers as described, in view of the tumor to be detected.

The reagents may be tagged or labeled with reagents capable of providing a detectable signal, depending upon the assay format employed. Such labels are capable, alone or in concert with other compositions or compounds, of providing a detectable signal. Most desirably, the label is detectable visually, e.g., colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods described herein are detectable by other means, e.g., colored latex microparticles (Bangs Laboratories, Indiana). Still other labels include fluorescent compounds, radioactive compounds or elements. Preferably, a reagent is associated with, or conjugated to a fluorescent detectable fluorochromes, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon assay method. Detectable labels for attachment to antibodies useful in methods described herein may be easily selected from among numerous compositions known and readily available to one skilled in the art of diagnostic assays.

Provided herein, in one aspect, is a method of diagnosing cancer in a subject. The method includes contacting a red blood cell-containing sample (as described herein) from the subject with a reagent capable of detecting a tumor-associated molecule in the sample and diagnosing the subject with cancer when tumor-associated molecule is detected in the sample. In one embodiment, the sample is substantially free from all other blood components other than RBCs (e.g., plasma, white blood cells, platelets, etc.) In one embodiment, the sample volume is about 1 μL to about 10 μL.

In some embodiments, DNA is extracted from the RBC containing sample. Methods for DNA extraction are known in the art. For example, the sample is centrifuged and the supernatant is discarded. A 20 mg/mL lysozyme solution (20 mM Tris-HCl, pH 8.0/2 mM EDTA, 1.2% Triton X-100) is added to the resulting precipitate at 37° C. Lysis treatment is performed for 30 minutes. Furthermore, a DNA extract can be obtained by performing column purification. This DNA extract may be used as a sample for PCR.

In some embodiments, the DNA is then subjected to amplification by PCR. Thus, in certain embodiments, the reagent capable of detecting a tumor-associated molecule in the sample includes PCR primers. Using the extracted DNA and primers, the gene region targeted for amplification is amplified by PCR, using primers known in the art, and as described herein.

Specifically, by using a primer set consisting of a forward primer and a reverse primer, e.g., the region of expected genetic mutation can be amplified. As the PCR reaction solution, for example, a nucleic acid synthesis substrate, a primer set, a nucleic acid synthase, a sample DNA, a buffer solution, and a solution containing water as the remaining components can be suitably used. Similar embodiments are contemplated for other genes as described herein. For example, to identify KRAS mutations, primers that amplify the relevant portion of Exon 2, Exon 3, and/or Exon 4 may be used, such as those described by Guarnaccia et al, 2018, cited above.

TABLE 4
Exemplary KRAS primers

| | | | Product | Basic |
|---|---|---|---|---|
| Name | Primer F | Primer R | Size | Tm |
| KRAS | ACTGGTGGAGTATTTGATAGT | AGAATGGTCCTGCACCAGTA | 249 | 52 °C |
| exon2 | GTAT (SEQ ID NO: 1) | A (SEQ ID NO: 2) | | |
| KRAS | AGGTGCACTGTAATAATCCAG | AACCCACCTATAATGGTGAA | 228 | 53 °C |
| exon3 | ACT (SEQ ID NO: 3) | TATCT (SEQ ID NO: 4) | | |
| KRAS | AAGGACTCTGAAGATGTACCT | AGAAGCAATGCCCTCTCAAG | 293 | 53 °C |
| exon4 | ATG (SEQ ID NO: 5) | (SEQ ID NO: 6) | | |

In certain embodiments, the reagent capable of detecting a tumor-associated molecule in the sample includes an oligonucleotide probe. In a hybridization-based method, probe design is a crucial step: the efficiency depends on a strong affinity between the specific target and the short oligonucleotide probes. In, for example, a gene chip format, multiple (up to thousands) of specific DNA probes are provided that can bind to mutation regions of the DNA sequence being analyzed. Each probe is a short sequence of DNA that is complementary to a specific region of the DNA being analyzed. Exemplary probes relating to KRAS mutations are found in Table 5 below, as described by Guarnaccia et al, 2018, cited above. SEQ ID NOs. 7-50 are provided in descending order.

TABLE 5
Exemplary KRAS probes

| | | Probe Sequences (5′-3′) | | |
|---|---|---|---|---|
| KRAS | Probe Name | with C6-NH2 Mod | Probe Type | Tm |
| EXON 2 | [symbol] | [symbol] | Wild-Type CAPTURE PROBE | [symbol] |
| | [symbol] | [symbol] | Mutant CAPTURE PROBE | [symbol] |
| | [symbol] | [symbol] | Mutant CAPTURE PROBE | [symbol] |
| | [symbol] | [symbol] | Mutant CAPTURE PROBE | [symbol] |
| | [symbol] | [symbol] | Wild-Type CAPTURE PROBE | [symbol] |
| | [symbol] | [symbol] | Mutant CAPTURE PROBE | [symbol] |
| | [symbol] | [symbol] | Mutant CAPTURE PROBE | [symbol] |
| | [symbol] | [symbol] | Mutant CAPTURE PROBE | [symbol] |
| | [symbol] | [symbol] | Wild-Type CAPTURE PROBE | [symbol] |
| | [symbol] | [symbol] | Mutant CAPTURE PROBE | [symbol] |
| | [symbol] | [symbol] | Mutant CAPTURE PROBE | [symbol] |
| | [symbol] | [symbol] | Mutant CAPTURE PROBE | [symbol] |
| | [symbol] | [symbol] | Wild-Type CAPTURE PROBE | [symbol] |
| | [symbol] | [symbol] | Mutant CAPTURE PROBE | [symbol] |
| | [symbol] | [symbol] | Mutant CAPTURE PROBE | [symbol] |
| | [symbol] | [symbol] | Mutant CAPTURE PROBE | [symbol] |
| EXON 3 | [symbol] | [symbol] | Wild-Type CAPTURE PROBE | [symbol] |
| | [symbol] | [symbol] | Mutant CAPTURE PROBE | [symbol] |
| | [symbol] | [symbol] | Mutant CAPTURE PROBE | [symbol] |
| | [symbol] | [symbol] | Mutant CAPTURE PROBE | [symbol] |
| | [symbol] | [symbol] | Wild-Type CAPTURE PROBE | [symbol] |
| | [symbol] | [symbol] | Mutant CAPTURE PROBE | [symbol] |
| | [symbol] | [symbol] | Wild-Type CAPTURE PROBE | [symbol] |
| | [symbol] | [symbol] | Mutant CAPTURE PROBE | [symbol] |
| | [symbol] | [symbol] | Mutant CAPTURE PROBE | [symbol] |
| | [symbol] | [symbol] | Wild-Type CAPTURE PROBE | [symbol] |
| | [symbol] | [symbol] | Mutant CAPTURE PROBE | [symbol] |
| EXON 4 | [symbol] | [symbol] | Wild-Type CAPTURE PROBE | [symbol] |
| | [symbol] | [symbol] | Wild-Type CAPTURE PROBE | [symbol] |
| | [symbol] | [symbol] | Mutant CAPTURE PROBE | [symbol] |
| | [symbol] | [symbol] | Mutant CAPTURE PROBE | [symbol] |
| | [symbol] | [symbol] | Mutant CAPTURE PROBE | [symbol] |
| | [symbol] | [symbol] | Mutant CAPTURE PROBE | [symbol] |
| | [symbol] | [symbol] | Wild-Type CAPTURE PROBE | [symbol] |
| | [symbol] | [symbol] | Mutant CAPTURE PROBE | [symbol] |
| | [symbol] | [symbol] | Mutant CAPTURE PROBE | [symbol] |
| | [symbol] | [symbol] | Wild-Type CAPTURE PROBE | [symbol] |
| | [symbol] | [symbol] | Mutant CAPTURE PROBE | [symbol] |
| | [symbol] | [symbol] | Hybridization control | |
| | [symbol] | [symbol] | Hybridization control | |
| | [symbol] | [symbol] | Hybridization control | |
| | [symbol] | [symbol] | Negative control | |
| | BG1 | [symbol] | PCR control | |
| | BG2 | [symbol] | PCR control | |

[symbol]

indicates data missing or illegible when filed

The prepared DNA sample is then added to the gene chip and allowed to hybridize, or bind, to the DNA probes on the chip. If the DNA in the sample contains a mutation or variation in the sequence, it will not hybridize to the corresponding probe on the chip. When the presence or absence of an amplification product by PCR is specified by a DNA chip, a label is added to the amplification product. The labeling method is not particularly limited, but a fluorescent label can be preferably used. When fluorescent labeling is performed by PCR, an amplification product in which only the ends are labeled can be generated using a fluorescently labeled primer. In addition, an amplification product containing a label therein can also be generated using a fluorescently labeled nucleic acid synthesis substrate. In any case, Cy5 or Cy3 can be suitably used as the fluorescent labeling component. Furthermore, as a label, it is also possible to use a label other than fluorescence, such as digoxigenin, biotin, and a radioisotope.

Moreover, a general thermal cycler etc. can be used as an apparatus which performs PCR reaction. The reaction conditions for PCR can be performed, for example, as follows. (A) 94° C. 2 minutes, (b) 94° C. (DNA denaturation step) 30 seconds, (c) 60° C. (annealing step) 30 seconds, (d) 72° C. (DNA synthesis step) 60 seconds ((b) to (D) 35 cycles), (e) 72° C. 3 minutes. These conditions are provided as an example and not intended to limit the invention.

In certain embodiments, it is desirable to use a DNA chip in order to specifically identify a plurality of tumor-associated molecules simultaneously in one system. It is preferable to use a DNA chip on which a sequence complementary to a probe sequence is immobilized. These probes each have a specific sequence for each target molecule, and can hybridize only with the amplification product of the corresponding gene region, so that each target molecule to be tested can be specifically detected simultaneously.

The DNA chip can be produced by an existing general method using the above probe. For example, when an affixed type DNA chip is produced, the probe can be immobilized on a glass substrate by a DNA spotter and a spot corresponding to each probe can be formed. When a synthetic DNA chip is produced, it can be produced by synthesizing a single-stranded oligo DNA having the above sequence on a glass substrate by a photolithography technique. Furthermore, the substrate is not limited to glass, and a plastic substrate, a silicon wafer, or the like can also be used. Further, the shape of the substrate is not limited to a flat plate shape, and may be various three-dimensional shapes, and a substrate having a functional group introduced so that a chemical reaction can be performed on the surface can be used.

### Treatment

In some embodiments of the methods described herein, the subject is treated cancer after being diagnosed with the same. In certain embodiments, follow-up testing, including DNA sequencing, biopsy, MRI, CAT scan, PET scan, etc. may be performed before or after performance of the method described herein.

In certain embodiments, treatment includes administration of a chemotherapeutic agent. Chemotherapeutic agents are compounds that exhibit anticancer activity and/or are detrimental to a cell (e.g., a toxin). Suitable chemotherapeutic agents for use in the methods disclosed herein include, but are not limited to: toxins (e.g., saporin, ricin, abrin, ethidium bromide, diptheria toxin, Pseudomonas exotoxin, and others listed above); alkylating agents (e.g., nitrogen mustards such as chlorambucil, cyclophosphamide, ifosfamide, mechlorethamine, melphalan, and uracil mustard; aziridines such as thiotepa; methanesulphonate esters such as busulfan; nitrosoureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). In one embodiment, the chemotherapeutic agent is selected from the group consisting of: placitaxel (Taxol®), cisplatin, docetaxol, carboplatin, vincristine, vinblastine, methotrexate, cyclophosphamide, CPT-11, 5-fluorouracil (5-FU), gemcitabine, estramustine, carmustine, adriamycin (doxorubicin), etoposide, arsenic trioxide, irinotecan, and epothilone derivatives.

In certain embodiments, treatment for cancer includes one or more of the following, optionally in addition to chemotherapy:

Radiation therapy: This treatment involves using high-energy radiation to kill cancer cells. It can be delivered externally or internally, and is often used in combination with surgery or chemotherapy.

Immunotherapy: This treatment uses the body's immune system to fight cancer. It works by stimulating the immune system to recognize and attack cancer cells. Some examples of immunotherapy include checkpoint inhibitors, CAR-T cell therapy, and cancer vaccines.

Targeted therapy: This treatment targets specific molecules or proteins that are involved in the growth and spread of cancer cells. Examples of targeted therapies include tyrosine kinase inhibitors and monoclonal antibodies.

Hormone therapy: This treatment is used for cancers that are hormone-sensitive, such as breast and prostate cancer. It works by blocking or reducing the production of hormones that stimulate the growth of cancer cells.

Surgery: This treatment involves removing cancerous tumors or tissue from the body. It is often used in combination with other treatments, such as radiation or chemotherapy.

Palliative care: This treatment focuses on managing symptoms and improving quality of life for patients with advanced or terminal cancer. It includes pain management, emotional support, and other forms of supportive care.

### Compositions and Kits

The compositions, kits and methods described herein include reagents which are capable of detecting, binding, specifically complexing with, or measuring the level of the tumor-associated molecule. Such reagents include those which are capable of detecting, or measuring the abundance of, said molecule at the nucleic acid level. Suitable reagents include those for detection by polymerase chain reaction (PCR). Suitable reagents can be purchased commercially,

In addition, suitable reagents may be designed by the person of skill in the art based on the published sequences of the specific tumor of interest. In one embodiment, the reagents are PCR primers and/or probes. In addition, other suitable components are included to allow for the identification and/or quantitation of the subject tumor. Such components include, e.g., enzymes, buffers and deoxynucleotides necessary for reverse transcription and/or PCR, preferably for qualitative and/or quantitative RT-PCR, detectable probes and/or an internal control.

Any combination of the described reagents for the detection of the subject tumor-associated molecule can be assembled in a diagnostic kit. For example, one embodiment of a diagnostic kit includes reagents for at least 2, 3, 4, 5, 6, 7, 8, 9, 10, 11, 12, 13, 14, 15, 16, 17, 18, 19, 20, 21, 22, 23, 24, 25, 30, 25, 40, 45, 50, 55, 60, 65, 70, 75, 80, 85, 90, 95, or 100 tumor-associated genetic mutations. In one embodiment, the kit includes reagents for 5 or 6 tumor-associated genetic mutations. In one embodiment, one or more of the reagents is associated or bound to a detectable label or bound to a substrate.

For these reagents, the labels may be selected from among many known diagnostic labels, including those described above. Similarly, the substrates for immobilization may be any of the common substrates, glass, plastic, a microarray, a microfluidics card, a chip or a chamber.

It is intended that any of the compositions described herein can be a kit containing multiple reagents or one or more individual reagents. For example, one embodiment of a composition includes a substrate upon which one or more of the reagents are immobilized. In another embodiment, the composition is a kit also contains optional detectable labels, immobilization substrates, optional substrates for enzymatic labels, as well as other laboratory items. In one embodiment, the kit contains a standard for use as a control.

The invention is now described with reference to the following examples. These examples are provided for the purpose of illustration only and the invention should in no way be construed as being limited to these examples but rather should be construed to encompass any and all variations that become evident as a result of the teaching provided herein.

### EXAMPLES

### Example 1: RBCs Acquire Tumor DNA from Lung Cancer Cells

RBCs transit through all tissues, continuously coming in contact with host and pathogen-derived mediators, including highly vascularized tumors. We recently discovered that RBCs sequester mitochondrial DNA through cell-surface expression of the receptor TLR9. Although we have not detected nuclear DNA bound to RBCs in the past (Hotz AJRCCM, supplement), we speculated that tumor DNA may be acquired by RBCs due to the highly vascular nature of tumors. We hypothesized that RBCs would sequester tumor DNA and that tumor DNA can be detected in less than 1 drop of RBCs.

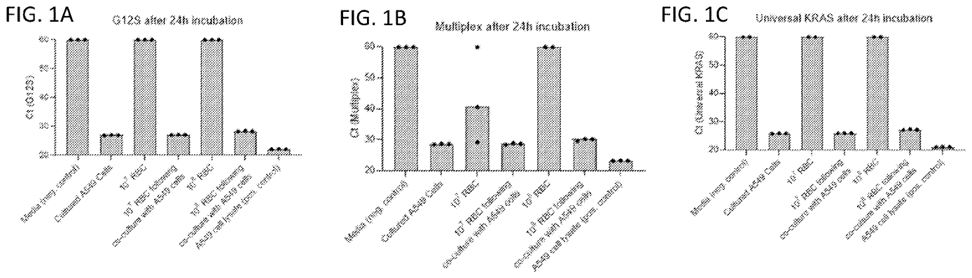

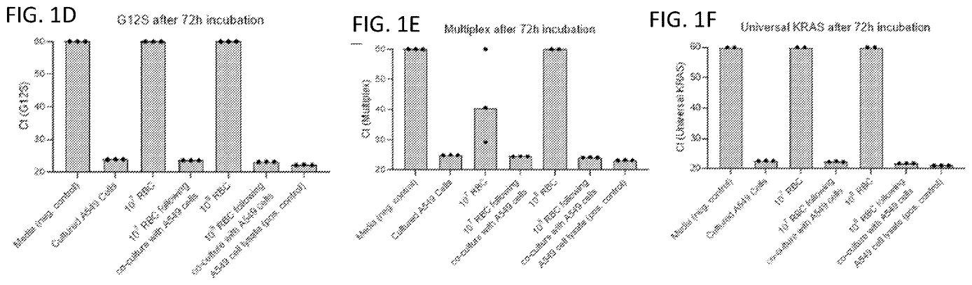

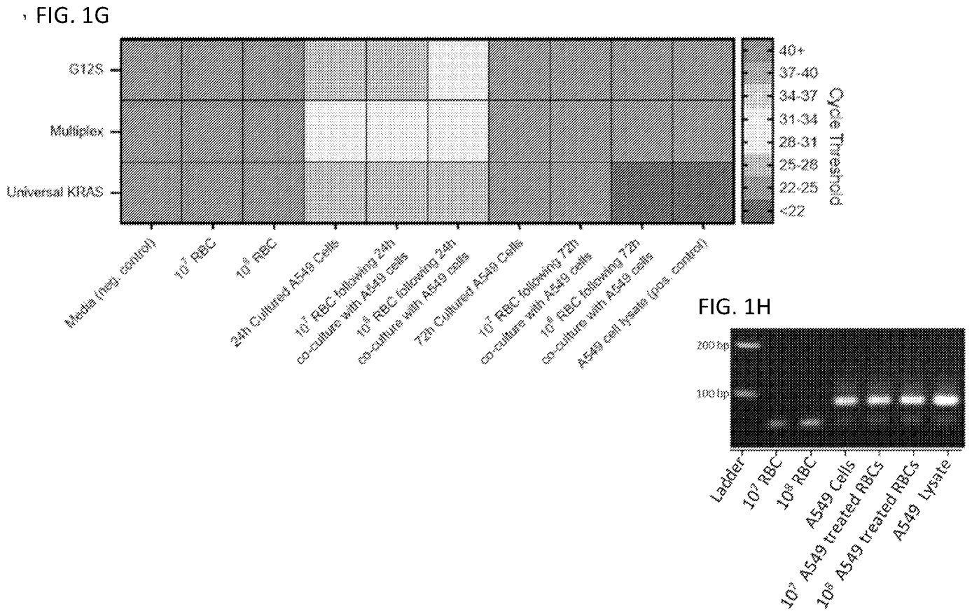

We first asked whether RBCs would acquire DNA from lung cancer cell lines that harbor KRAS mutations. As seen in FIG. 1, we can detect tumor DNA on RBCs co-cultured with tumor cells.

### Example 2: RBCs Acquire Mutant DNA from Cultured Tumor Cells

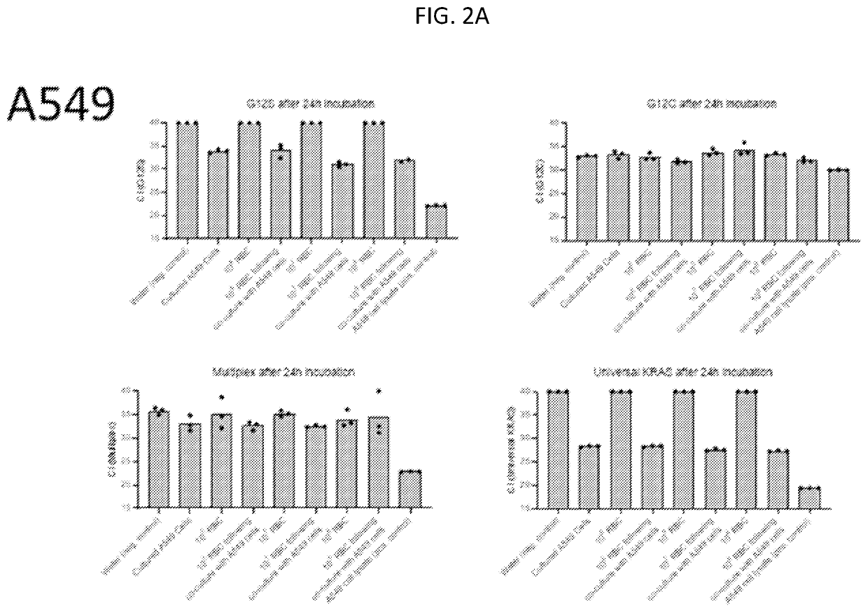

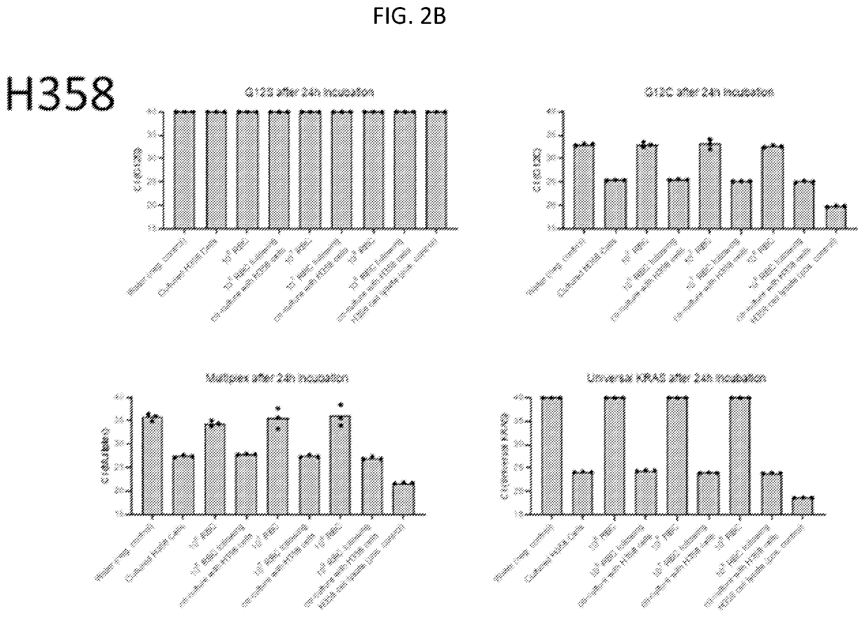

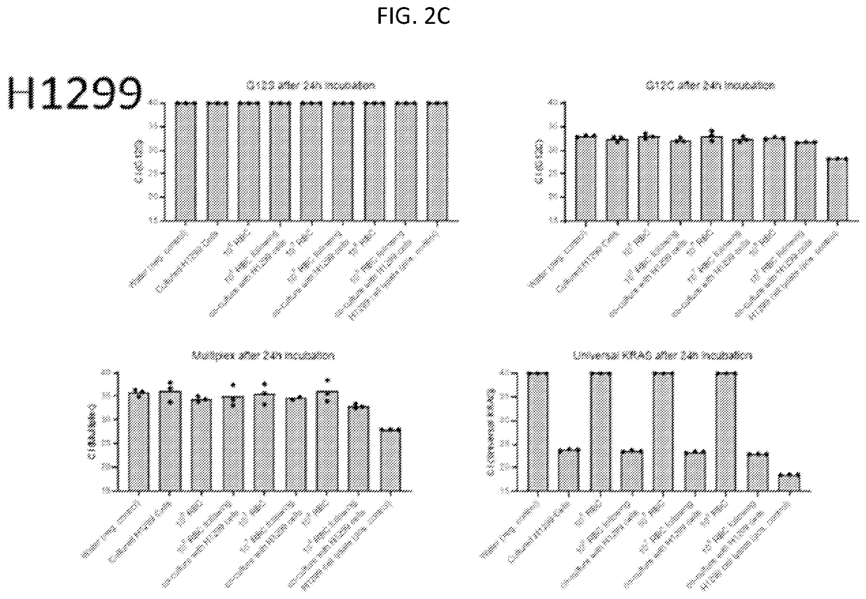

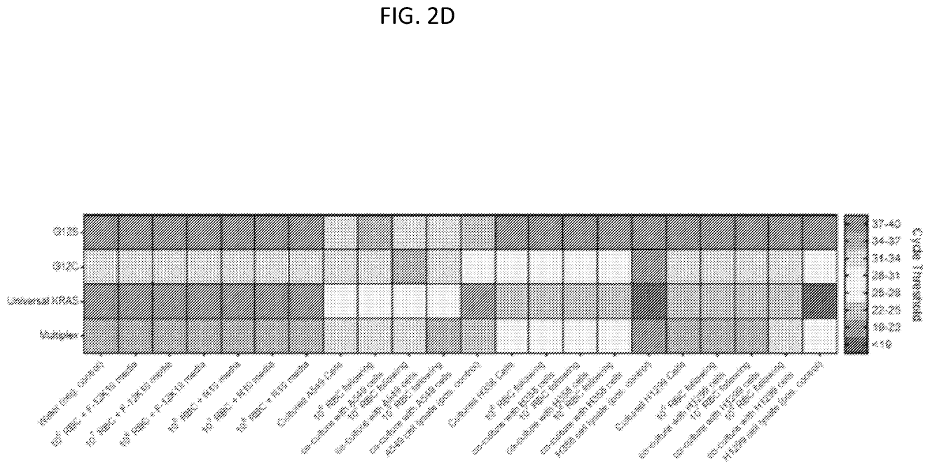

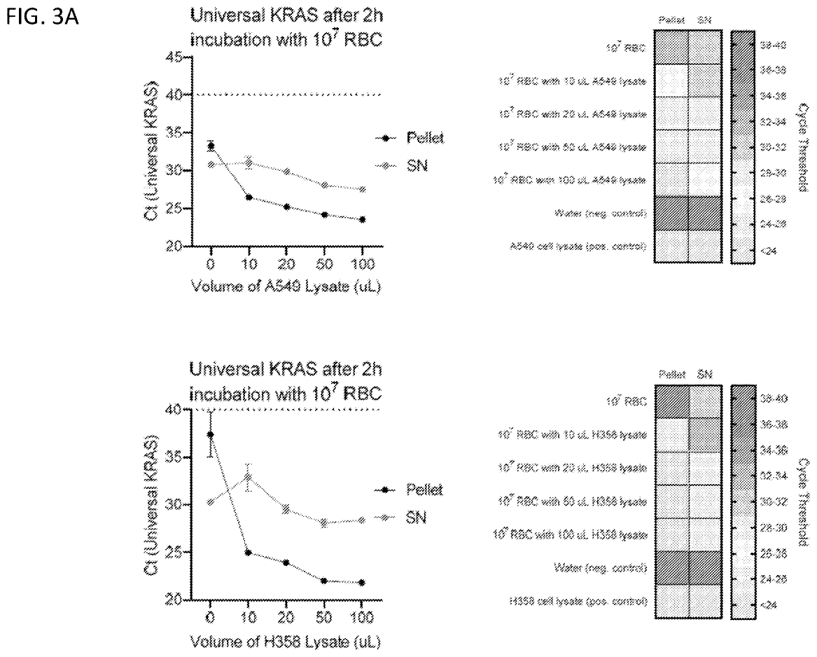

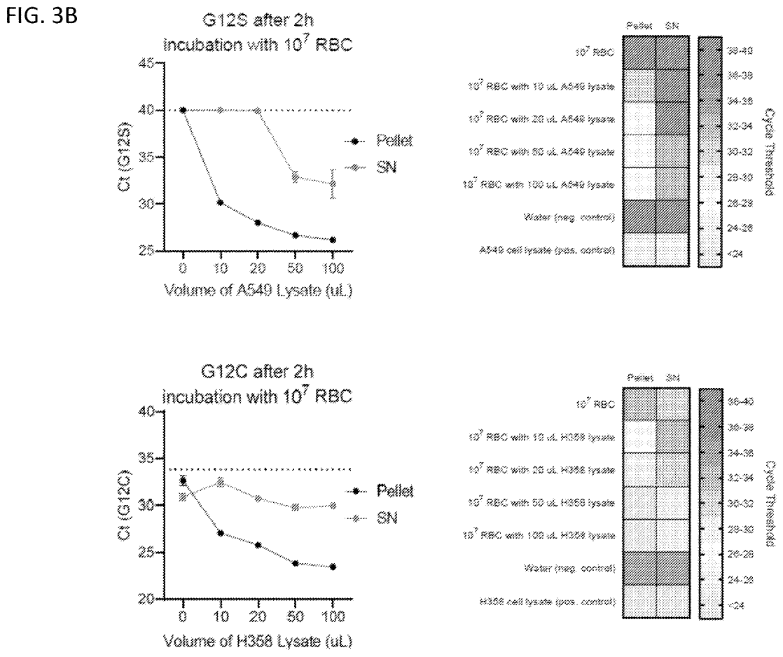

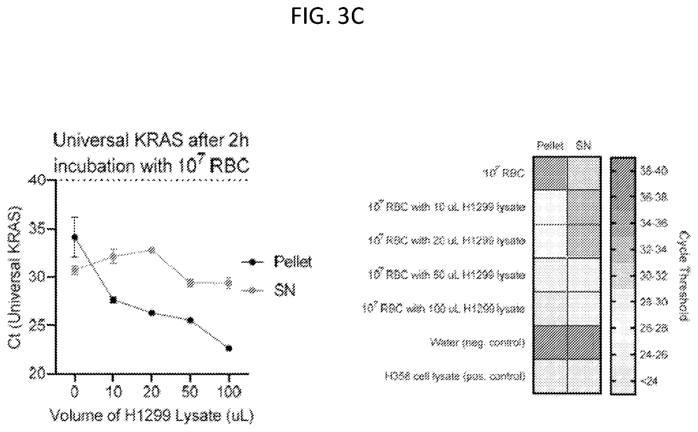

We next examined additional cell lines including the H358 cell line which harbors the KRAS G12C mutation and the H1299 cell line which does not harbor any KRAS mutations. Similar to FIG. 1, cells were seeded in 6 well plates prior to incubation with purified naïve RBCs. However, rather than growing to confluence for several days prior to RBC treatment, the tumor cells were only seeded overnight prior to the experiment. qPCR for the G12S and G12C KRAS mutants was performed on extracted DNA. As controls a mutant multiplex and universal KRAS PCR was also run. As shown in FIG. 2 the RBCs acquire mutant DNA from cultured tumor cells.

### Example 3: RBCs are Able to Detect and Bind Ultra-Low Amounts of Tumor Cell Lysate

We next asked if RBCs would acquire tumor DNA at extremely low doses of DNA, and if RBCs would sequester the tumor DNA from the plasma/SN. RBCs were incubated differing volumes of tumor cell lysate. The lysate was generated from 1 million tumor cells. As seen below the RBCs are able to detect and bind ultra-low amounts of tumor cell lysate.

### Example 4: tODN Loading of RBCs

RBCs from healthy donors (10⁷) will be incubated with fluorescently labeled synthetic tODNs at various doses (0 nM/1 million RBCs-100 nM tODN/1 million RBCs) for varying time points (30 minutes, 60 minutes, 120 minutes) at 37° C. with gentle agitation. We will use sequences found in the common somatic variants in lung cancer (KRAS G12C, EGFR L858R, and EGFR exon 19 deletions (synthesized by IDT)). RBC-bound mutant ODN, and supernatant mutant ODN will be compared using qPCR.

RBC-tODN binding will be determined using FACS as we have previously described for CpG ODN.

RBCs will be incubated with unlabeled tODN as specified above. Following incubation, the RBCs will be separated from the supernatant (SN) by spinning over a 30% sucrose gradient. qPCR on the SN and RBC-associated DNA will be performed to quantify bound versus unbound tODN. If qPCR is insensitive for detecting RBC-ODN binding, but we detect binding by flow cytometry, we will perform digital drop PCR on RBC and supernatants.

RBCs from patients with known oncogene-driven advanced NSCLC and NSCLC without oncogene-driven tumors and healthy donors will be isolated. Human erythrocytes will be obtained from whole blood and isolated using glycophorin A beads as described in the literature. PCR for the known somatic variants will be performed on RBCs and plasma from patients and control cancer patients (without driver mutations) and healthy controls. 15 patients/group will be analyzed in this pilot study.

### Example 5: RBCs Sequester Extracellular RNA, and Tumor RNA is Detectable from RBCs

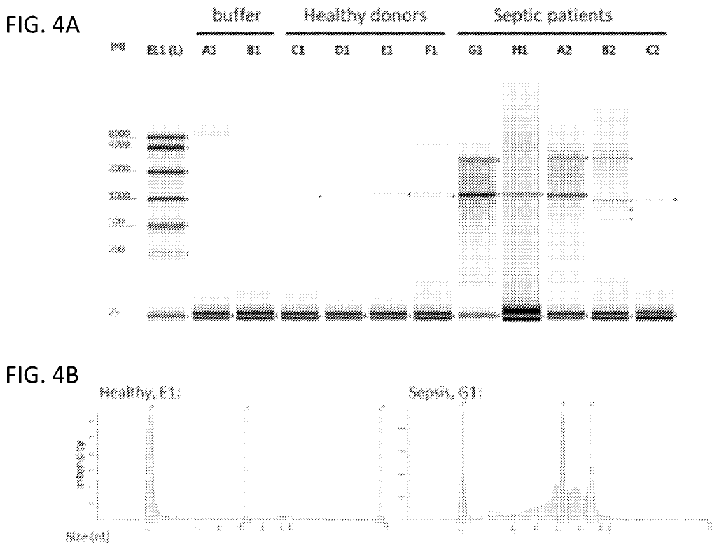

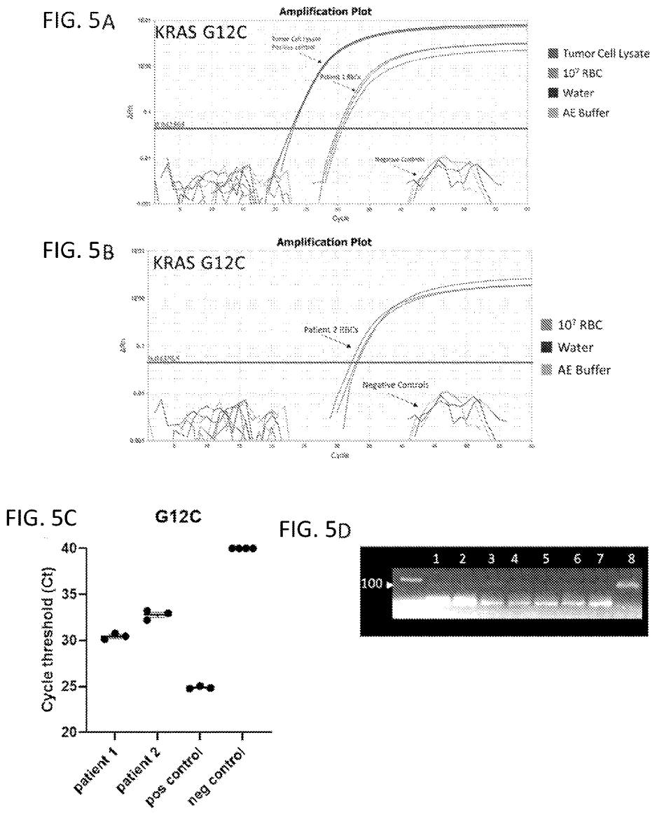

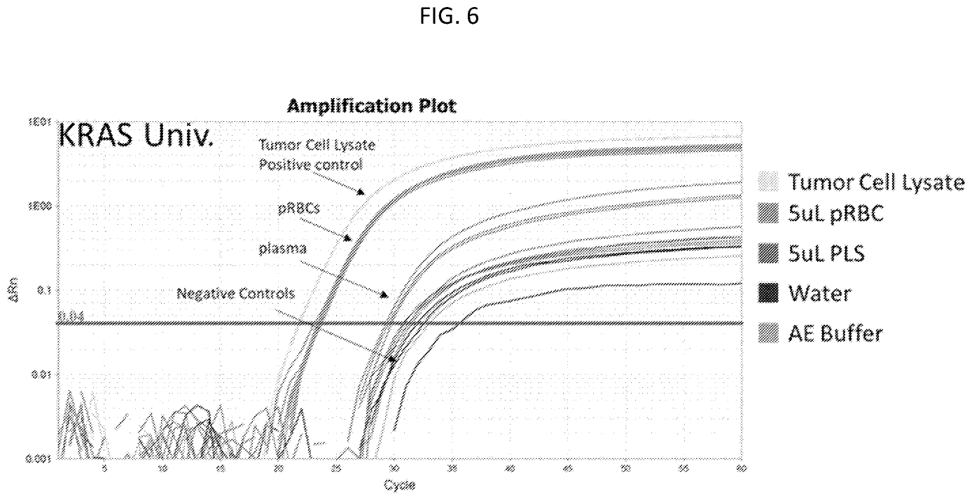

We recently asked whether RNA would be obtainable from RBCs from healthy donors and patients admitted to the hospital with an inflammatory state. As seen in FIG. 4, in contrast to controls, we detected a substantial amount of RNA on RBCs during an acute inflammatory syndrome (sepsis). Since RBCs do not synthesize new proteins or contain RNA, this RNA is acquired from other host or microbial sources. Because cancer patients are reported to have elevated cell-free nucleic acids, we hypothesized that RBCs from patients with cancer will contain a substantial amount of RNA and provide an opportunity to characterize circulating free RNA patterns in lung cancer.

### Example 6: Detection of HPV DNA on RBCs

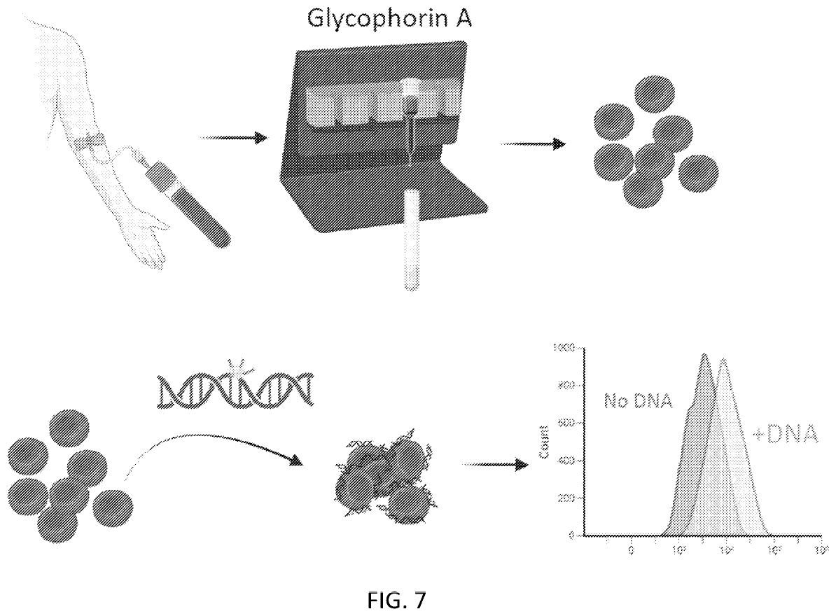

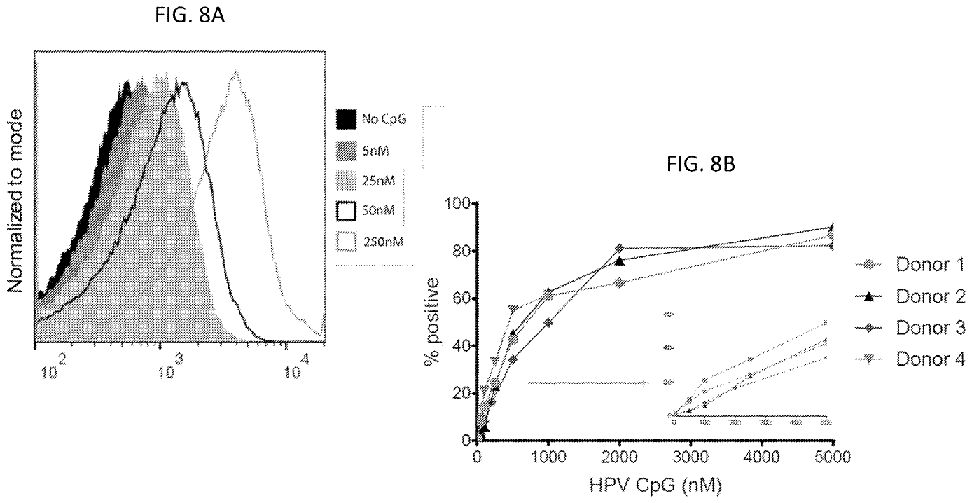

Next, we asked whether Human papillomavirus (HPV) DNA could be detected on RBCs from healthy donors and patients incubated with HPV DNA in vitro. As shown in FIG. 7, a FITC-labeled DNA motif from the E6 sequence of HPV16 was incubated with RBCs. The RBC-HPV DNA binding was then assessed by flow cytometry. FIG. 8A shows the binding of RBCs from a donor to HPV DNA after incubation with 0 nM, 5 nM, 25 nM, 50 nM, or 250 nM of HPV CpG. As the dose of HPV CpG increases, the amount of RBCs that bind the HPV DNA also increases. Additionally, RBCs from 4 donors were incubated with a broader range of HPV CpG doses in FIG. 8B. This figure confirms that the percentage of cells that bind to HPV DNA increase as the dose increases with ~80% of cells being positive for HPV DNA after incubation with 2000 nM HPV CpG.

### Example 7: Detection of HPV DNA from Cancer Cells on RBCs

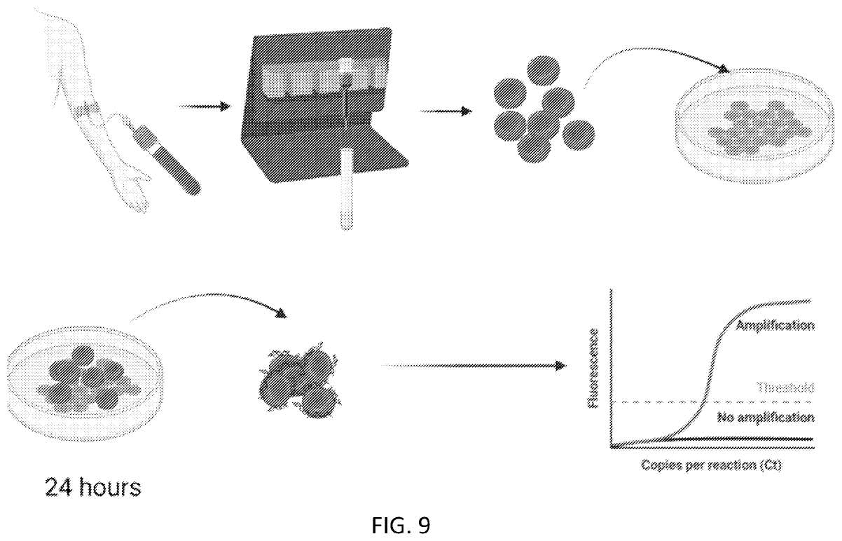

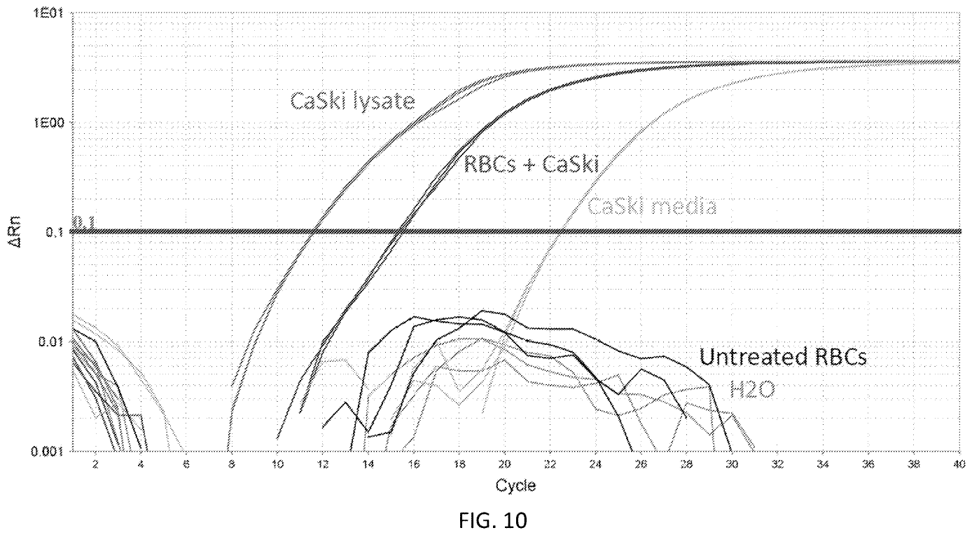

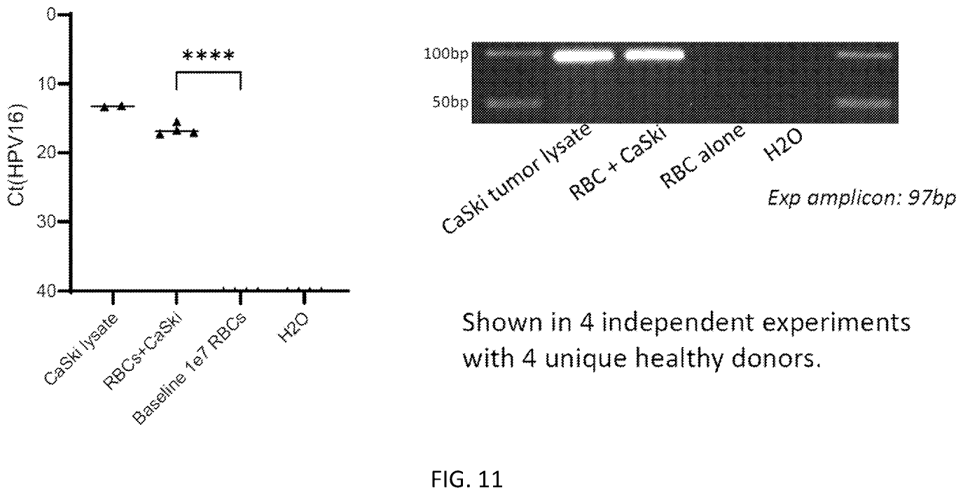

Next, we asked whether RBCs could acquire HPV DNA from cervical cancer cell lines infected with HPV. As shown in FIG. 9, RBCs were incubated for 24 hours with CaSki cells, a cancer cell line known to contain HPV16. DNA was then extracted from the RBCs and HPV16 DNA was detected by qPCR. An HPV16 qPCR amplification curve was prepared from this experiment (FIG. 10). This experiment was repeated in 4 independent experiments with 4 unique healthy RBC donors. The data from these experiments is provided in FIG. 11. Taken together, this data demonstrates that RBCs acquire HPV16 DNA from cervical cancer cell lines.

The data presented herein above can be applied clinically to patients as a diagnostic test for HPV and cervical cancer.

### Example 8: Detection of Cancer Cell HPV DNA on RBCs

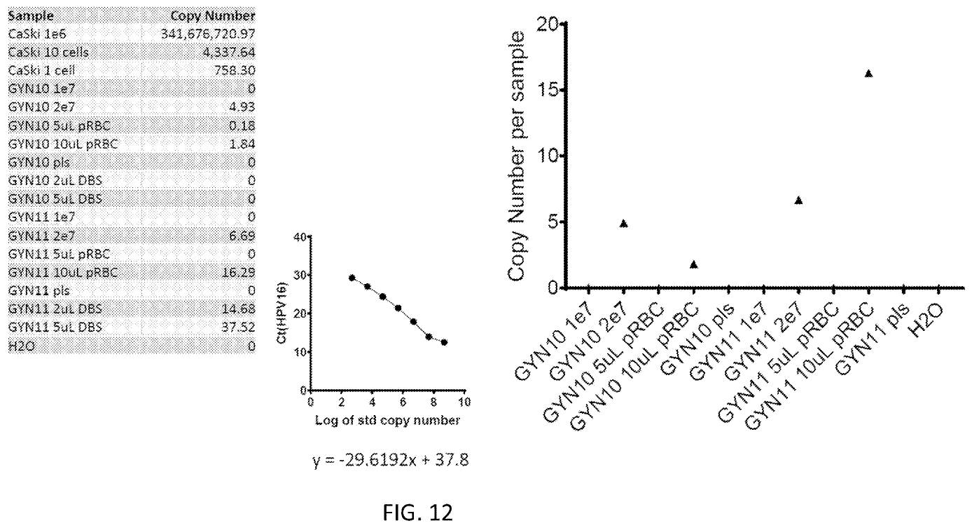

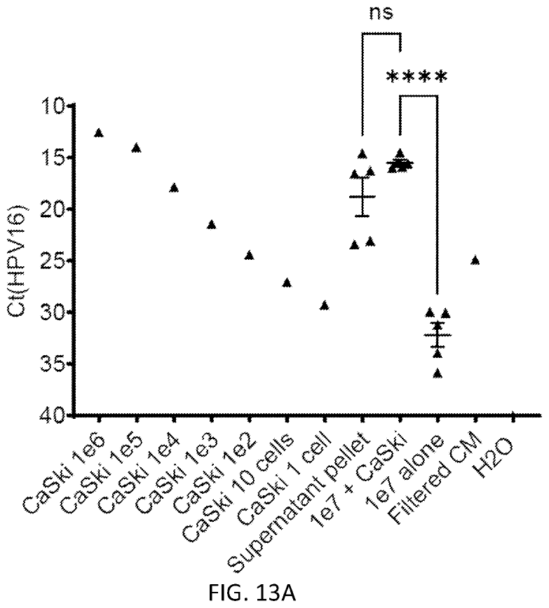

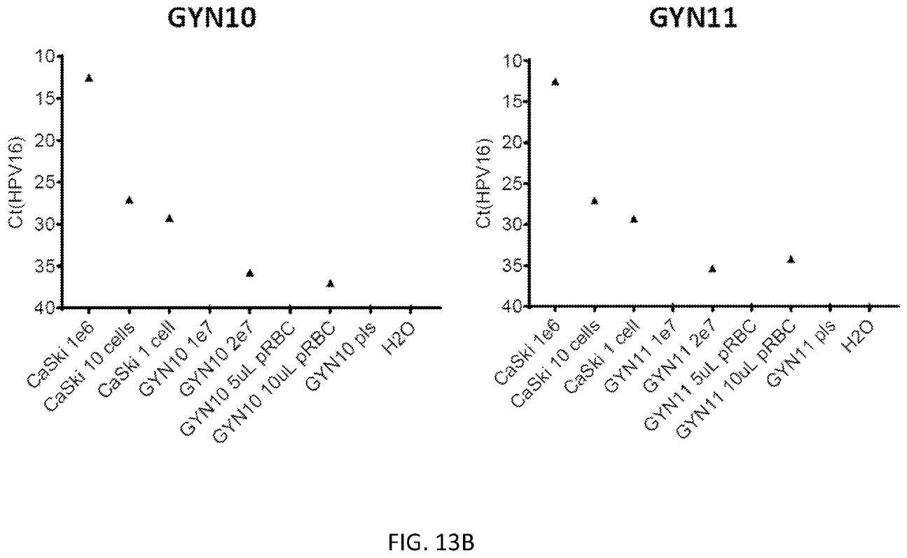

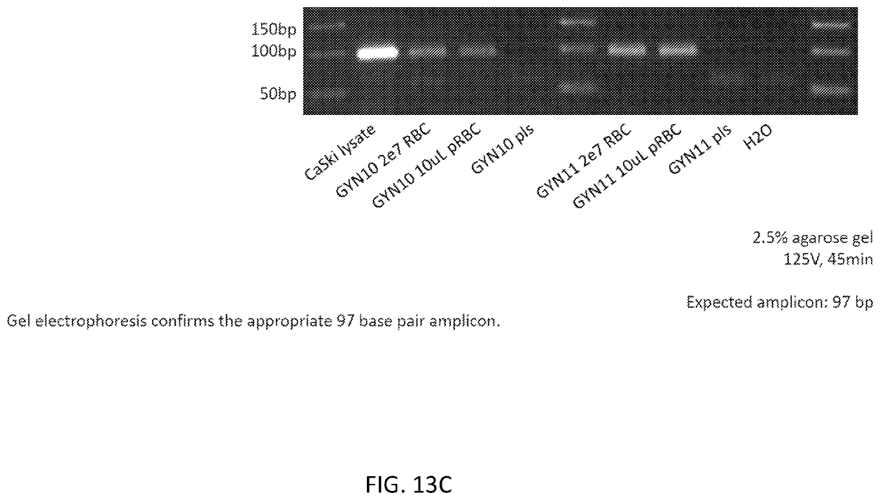

Next, we asked whether RBCs could acquire HPV DNA from other cervical cancer cell lines infected with HPV. To start, we detected the copy number for HPV16 DNA in various cell lines using PCR. (FIG. 12) Next, the cells from patients with locally advanced HPV16+ cervical cancer were incubated with RBCs. In both patients, HPV16 DNA was detected on 2e7 RBCs and 10 uL pRBCs. (FIG. 13B) These results were confirmed using gel electrophoresis (FIG. 13C).

This was then repeated in 5 independent experiments across 4 healthy donors. (FIG. 13A). This data confirms that healthy donor RBCs acquire HPV DNA.

### Example 9: Mechanism of RBC Binding to CpG

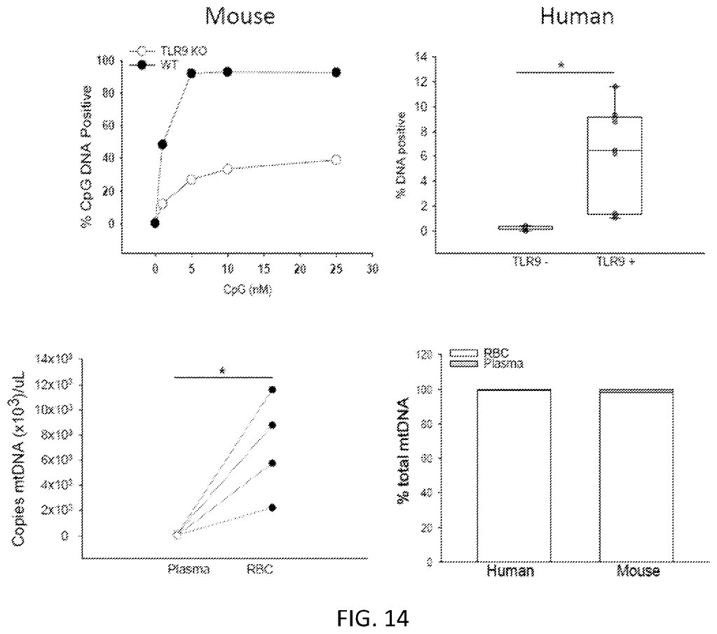

Next, we analyzed the mechanism by which RBCs bind and sequester CpG. This mechanism was analyzed by comparing DNA binding by RBCs in WT mice and TLR9KO mice. (FIG. 14) RBCs from the TLR9KO mice had a significantly lower % CpG DNA than RBCs from the wild type mice. This was confirmed in TLR9⁻ and TLR⁺ human cells. The location of the circulating DNA was also confirmed by comparing the amount of cfDNA in the plasma to the amount of cfDNA in the RBCs. In both mice and humans, limited cfDNA was located in the plasma. This indicates that RBVs preferentially sequester circulating DNA.

### Embodiments

1. A method of diagnosing cancer in a subject, the method comprising
contacting a red blood cell-containing sample from the subject with a reagent capable of detecting a tumor-associated DNA molecule in the sample; and
diagnosing the subject with a cancer when the tumor-associated DNA molecule is detected in the sample.

2. A method of diagnosing cancer in a subject, the method comprising
contacting a red blood cell-containing sample from the subject with a reagent capable of detecting a tumor-associated molecule in the sample; and
diagnosing the subject with a cancer when the tumor-associated molecule is detected in the sample.

3. The method according to embodiment 1 or 2, wherein the reagent is specific to a tumor-associated genetic mutation.

4. The method according to any one of embodiments 1 to 3, wherein the cancer is a cancer of the lung, cervix, breast, prostate, thyroid, colon, or pancreas.

5. The method according to embodiment 1, wherein the reagent comprises multiple reagents, each capable of detecting a different specific tumor-associated molecule.

6. The method according any preceding embodiment, wherein the reagent comprises primers capable of binding tumor-associated DNA or RNA.

7. The method according to any preceding embodiment, wherein the specific tumor-associated genetic mutation found in the sample is identified.

8. The method according to any preceding embodiment, wherein the tumor-associated genetic mutation is found in the EGFR, ALK, KRAS, ROS1, BRAF, NTRK, MET, RET, ERBB2, PIK3CA, BRCA1, BRCA2, FGFR2, FGFR3, EZH2, or PDGFRA gene.

9. The method according to embodiment 8, wherein the tumor-associated genetic mutation is found in the KRAS gene.

10. The method according to any preceding embodiment, wherein the method further comprises treating the subject for cancer, when diagnosed with the same.

11. The method according to embodiment 10, wherein the treatment is specific to the identified tumor-associated genetic mutation.

12. The method according to any preceding embodiment, wherein the diagnostic method is performed at the point of care.

13. The method according to any preceding embodiment, further comprising filtering the blood sample to remove non-RBC blood cells.

14. The method according to any one of embodiments 1 to 13, wherein the sample volume is 10 uL or less.

15. The method according embodiment 14, wherein the sample volume is from about 1 uL to about 5 ul.

16. The method according to any preceding embodiment, wherein the sample contains at least 1 million RBC.

17. The method according to any preceding embodiment, wherein the RBCs are enriched from the sample.

18. The method according to any preceding embodiment, wherein the sample is substantially free of other blood components.

19. The method according to any preceding embodiment, wherein the subject is suspected of having cancer.

20. Use of a reagent capable of detecting a tumor-associated DNA molecule in a biological sample containing red-blood cells for diagnosing the subject with a cancer when the tumor-associated DNA molecule is detected in the sample.

1. A method of diagnosing cancer in a subject, the method comprising
contacting a red blood cell-containing sample from the subject with a reagent capable of detecting a tumor-associated DNA molecule in the sample; and
diagnosing the subject with a cancer when the tumor-associated DNA molecule is detected in the sample.

2. A method of diagnosing cancer in a subject, the method comprising
contacting a red blood cell-containing sample from the subject with a reagent capable of detecting a tumor-associated molecule in the sample; and
diagnosing the subject with a cancer when the tumor-associated molecule is detected in the sample.

3. The method according to claim 1, wherein the reagent is specific to a tumor-associated genetic mutation.

4. The method according to claim 1, wherein the cancer is a cancer of the lung, cervix, breast, prostate, thyroid, colon, or pancreas.

5. The method according to claim 1, wherein the reagent comprises multiple reagents, each capable of detecting a different specific tumor-associated molecule.

6. The method according to claim 1, wherein the reagent comprises primers capable of binding tumor-associated DNA or RNA.

7. The method according to claim 1, wherein the specific tumor-associated genetic mutation found in the sample is identified.

8. The method according to claim 1, wherein the tumor-associated genetic mutation is found in the EGFR, ALK, KRAS, ROS1, BRAF, NTRK, MET, RET, ERBB2, PIK3CA, BRCA1, BRCA2, FGFR2, FGFR3, EZH2, or PDGFRA gene.

9. The method according to claim 8, wherein the tumor-associated genetic mutation is found in the KRAS gene.

10. The method according to claim 1, wherein the method further comprises treating the subject for cancer, when diagnosed with the same.

11. The method according to claim 10, wherein the treatment is specific to the identified tumor-associated genetic mutation.

12. The method according to claim 1, wherein the diagnostic method is performed at the point of care.

13. The method according to claim 1, further comprising filtering the blood sample to remove non-RBC blood cells.

14. The method according to claim 1, wherein the sample volume is 10 uL or less.

15. The method according claim 14, wherein the sample volume is from about 1 uL to about 5 ul.

16. The method according to claim 1, wherein the sample contains at least 1 million RBC.

17. The method according to claim 1, wherein the RBCs are enriched from the sample.

18. The method according to claim 1, wherein the sample is substantially free of other blood components.

19. The method according to claim 1, wherein the subject is suspected of having cancer.

20. Use of a reagent capable of detecting a tumor-associated DNA molecule in a biological sample containing red-blood cells for diagnosing the subject with a cancer when the tumor-associated DNA molecule is detected in the sample.


SEQUENCE LISTING (50 sequences)

SEQ ID NO: 1 - RNA, 25 nt, synthetic construct
  source 1..25: mol_type = other RNA
actggtggagtatttgatagtgtat

SEQ ID NO: 2 - RNA, 21 nt, synthetic construct
  source 1..21: mol_type = other RNA
agaatggtcctgcaccagtaa

SEQ ID NO: 3 - RNA, 24 nt, synthetic construct
  source 1..24: mol_type = other RNA
aggtgcactgtaataatccagact

SEQ ID NO: 4 - RNA, 25 nt, synthetic construct
  source 1..25: mol_type = other RNA
aacccacctataatggtgaatatct

SEQ ID NO: 5 - RNA, 24 nt, synthetic construct
  source 1..24: mol_type = other RNA
aaggactctgaagatgtacctatg

SEQ ID NO: 6 - RNA, 20 nt, synthetic construct
  source 1..20: mol_type = other RNA
agaagcaatgccctctcaag

SEQ ID NO: 7 - RNA, 25 nt, synthetic construct
  source 1..25: mol_type = other RNA
tttttttggtagttggagctggtgg

SEQ ID NO: 8 - RNA, 25 nt, synthetic construct
  source 1..25: mol_type = other RNA
ttttttttagttggagcttgtggcg

SEQ ID NO: 9 - RNA, 25 nt, synthetic construct
  source 1..25: mol_type = other RNA
ttttttttagttggagctcgtggcg

SEQ ID NO: 10 - RNA, 25 nt, synthetic construct
  source 1..25: mol_type = other RNA
tttttttagttggagctagtggcgt

SEQ ID NO: 11 - RNA, 25 nt, synthetic construct
  source 1..25: mol_type = other RNA
tttttttggtagttggagctggtgg

SEQ ID NO: 12 - RNA, 25 nt, synthetic construct
  source 1..25: mol_type = other RNA
tttttttagttggagctgttggcgt

SEQ ID NO: 13 - RNA, 25 nt, synthetic construct
  source 1..25: mol_type = other RNA
tttttttagttggagctgctggcgt

SEQ ID NO: 14 - RNA, 25 nt, synthetic construct
  source 1..25: mol_type = other RNA
tttttttagttggagctgatggcgt

SEQ ID NO: 15 - RNA, 25 nt, synthetic construct
  source 1..25: mol_type = other RNA
ttttttttagttggagctggtggcg

SEQ ID NO: 16 - RNA, 25 nt, synthetic construct
  source 1..25: mol_type = other RNA
tttttttagttggagctggttgcgt

SEQ ID NO: 17 - RNA, 25 nt, synthetic construct
  source 1..25: mol_type = other RNA
ttttttttagttggagctggtcgcg

SEQ ID NO: 18 - RNA, 25 nt, synthetic construct
  source 1..25: mol_type = other RNA
tttttttagttggagctggtagcgt

SEQ ID NO: 19 - RNA, 25 nt, synthetic construct
  source 1..25: mol_type = other RNA
ttttttttagttggagctggtggcg

SEQ ID NO: 20 - RNA, 25 nt, synthetic construct
  source 1..25: mol_type = other RNA
tttttttagttggagctggtgacgt

SEQ ID NO: 21 - RNA, 25 nt, synthetic construct
  source 1..25: mol_type = other RNA
tttttttagttggagctggtgccgt

SEQ ID NO: 22 - RNA, 25 nt, synthetic construct
  source 1..25: mol_type = other RNA
tttttttagttggagctggtgtcgt

SEQ ID NO: 23 - RNA, 26 nt, synthetic construct
  source 1..26: mol_type = other RNA
tttttttattctcgacacagcaggtc

SEQ ID NO: 24 - RNA, 27 nt, synthetic construct
  source 1..27: mol_type = other RNA
tttttttggatattctcgacacagcag

SEQ ID NO: 25 - RNA, 26 nt, synthetic construct
  source 1..26: mol_type = other RNA
tttttttattctcgacacaacaggtc

SEQ ID NO: 26 - RNA, 27 nt, synthetic construct
  source 1..27: mol_type = other RNA
tttttttggatattctcgacacaacag

SEQ ID NO: 27 - RNA, 26 nt, synthetic construct
  source 1..26: mol_type = other RNA
tttttttcgacacagcaggtcaagag

SEQ ID NO: 28 - RNA, 26 nt, synthetic construct
  source 1..26: mol_type = other RNA
tttttttctcgacacagcaggtaaag

SEQ ID NO: 29 - RNA, 25 nt, synthetic construct
  source 1..25: mol_type = other RNA
tttttttacagcaggtcaagaggag

SEQ ID NO: 30 - RNA, 25 nt, synthetic construct
  source 1..25: mol_type = other RNA
tttttttcgacacagcaggtctaga

SEQ ID NO: 31 - RNA, 25 nt, synthetic construct
  source 1..25: mol_type = other RNA
tttttttacagcaggtcgagaggag

SEQ ID NO: 32 - RNA, 25 nt, synthetic construct
  source 1..25: mol_type = other RNA
tttttttacagcaggtcaagaggag

SEQ ID NO: 33 - RNA, 25 nt, synthetic construct
  source 1..25: mol_type = other RNA
tttttttcgacacagcaggtcatga

SEQ ID NO: 34 - RNA, 27 nt, synthetic construct
  source 1..27: mol_type = other RNA
tttttttaaatgtgatttgccttctag

SEQ ID NO: 35 - RNA, 27 nt, synthetic construct
  source 1..27: mol_type = other RNA
tttttttataaatgtgatttgccttct

SEQ ID NO: 36 - RNA, 27 nt, synthetic construct
  source 1..27: mol_type = other RNA
tttttttaactgtgatttgccttctag

SEQ ID NO: 37 - RNA, 27 nt, synthetic construct
  source 1..27: mol_type = other RNA
tttttttataactgtgatttgccttct

SEQ ID NO: 38 - RNA, 27 nt, synthetic construct
  source 1..27: mol_type = other RNA
tttttttaattgtgatttgccttctag

SEQ ID NO: 39 - RNA, 27 nt, synthetic construct
  source 1..27: mol_type = other RNA
tttttttataattgtgatttgccttct

SEQ ID NO: 40 - RNA, 27 nt, synthetic construct
  source 1..27: mol_type = other RNA
tttttttcagcaaagacaagacaggta

SEQ ID NO: 41 - RNA, 27 nt, synthetic construct
  source 1..27: mol_type = other RNA
tttttttatcaccaaagacaagacagg

SEQ ID NO: 42 - RNA, 27 nt, synthetic construct
  source 1..27: mol_type = other RNA
tttttttatcaacaaagacaagacagg

SEQ ID NO: 43 - RNA, 27 nt, synthetic construct
  source 1..27: mol_type = other RNA
tttttttatcagcaaagacaagacagg

SEQ ID NO: 44 - RNA, 27 nt, synthetic construct
  source 1..27: mol_type = other RNA
tttttttcagtaaagacaagacaggta

SEQ ID NO: 45 - RNA, 25 nt, synthetic construct
  source 1..25: mol_type = other RNA
agtgagggaggagatggaaccatct

SEQ ID NO: 46 - RNA, 25 nt, synthetic construct
  source 1..25: mol_type = other RNA
cacaacacaagtacctgacatggcg

SEQ ID NO: 47 - RNA, 25 nt, synthetic construct
  source 1..25: mol_type = other RNA
tggtcttcttaaaagattagtaggt

SEQ ID NO: 48 - RNA, 25 nt, synthetic construct
  source 1..25: mol_type = other RNA
ggcaaaggagctgctaagggatttc

SEQ ID NO: 49 - RNA, 21 nt, synthetic construct
  source 1..21: mol_type = other RNA
gcagagccatctattgcttac

SEQ ID NO: 50 - RNA, 21 nt, synthetic construct
  source 1..21: mol_type = other RNA
ctagggttggccaatctactc